(19) United States
(12) Patent Application Publication
(10) Pub. No.: US 2004/0221840 A1
(43) Pub. Date: Nov. 11, 2004

(54) Inhaler

(76) Inventor: Melissa Soyna Stockman-Lamb, Medford, NY (US)

Correspondence Address: Thomas A. O'Rourke, BODNER & O'ROURKE, L.L.P., SUITE 108, 425 BROADHOLLOW ROAD, MELVILLE, NY, 11747, US

(21) Appl. No.: 10/749,142

(22) Filed: Dec. 30, 2003

Related U.S. Application Data
(60) Provisional application No. 60/438,424, filed on Jan. 7, 2003.

(51) Int. Cl. A61M 11/00

(52) U.S. Cl. 128/200.23

(57) ABSTRACT

The present invention is directed to an apparatus that allows patients who use inhalers to keep track of the number of doses they have or have left of their medicine. The present invention prevents patients from unknowingly running out of medicine and enables patients to promptly get a refill of their medication.

This utility application claims priority on the U.S. provisional patent application Serial No. 60/438,424 filed on Jan. 7, 2003 the disclosures of which are incorporated herein by reference. The invention is an apparatus that allows for patients who take medicine through inhalers to keep track of the number of doses they have taken or have left of their medicine. The apparatus prevents patients from unknowingly running out of medicine, attempting to receive a dose of medicine, and getting nothing out of the inhaler. This is a very important consideration because patients may depend on the medicine to survive. The apparatus prevents this because the patient knows when the medicine is almost gone and can get a refill as appropriate or desired.

### FIELD OF THE INVENTION

### BACKGROUND OF THE INVENTION

One of the problems in the use of inhalers is the difficulty in determining how may doses remain in the inhaler. Since the inhaler contains many doses, typically in the order of 150 to 200 doses, it is very difficult for the average user to keep track of the number of doses taken. In addition, since the amount remaining in the inhaler is difficult to determine there is a serious risk of the patient running out of the medication at undesirable times. For example, a patient may go away for the weekend, or for along period of time during the day. There is a risk that the patient could run out. As a result, the patient may purchase a new inhaler before the original one is completed, in order to, be completely safe. Adding to the difficulty in keeping track of the uses, the patient may take one puff this time and four puffs next, may not use it for a month and then use it six times in a single day during allergy season or URI. Furthermore, canisters for inhalers contain varying numbers of doses. Currently, there is no convenient way for a patient to determine how man inhalations are used or remaining because it is nearly impossible to monitor how many doses have been utilized or remain. Also, when the patient asks for a prescription renewal sooner than anticipated, the physician may interpret this as overusing or overdosing i.e. steroid medicine, or non compliance with the prescription.

The prior art that exists includes a rotadisk sold under the trade name Advair. This apparatus requires fine motor coordination by the patient to be able to open the device. This is done by holding the device in one hand, and using the other hand to push a small lever (that opens a blister pack of powdered medicine and moves the small counter display to the next lowest number), simultaneously holding the device level while bringing it to the mouth and inhaling. It must be kept level during the inhalation to prevent the powder from escaping the chamber. If the unit is tilted, the powder will spill into the unit, the patient will not receive the medicine, and probably not realize they didn't receive the medicine until they begin to wheeze. The patient must then close the device. This requires using both hands, fine motor coordination, complex directions, and very good eyesight. This device may be difficult for patients with disabilities like osteo or rheumatoid arthritis, MD, post stroke, the young or aged because of the need for both hands and fine motor coordination. Furthermore, the very small numbers makes it difficult for patients with good eyesight to see, but especially difficult for those farsighted or who have cataracts or glaucoma. Deep inhalation by the patient activates the rotadisk. Sometimes, the medicine will not be effective because the patient may not be able to inhale deep enough to get the medicine to the lungs. In addition, if the medicine remains in the mouth, the steroids may increase the risk of developing thrush, a yeast infection in the mouth, which will require additional medicine.

The Advair device only applies to specific steroidal powder as a prophylaxis and does not apply to patients who need acute relief. The Advair can only keep count of 60 inhalations, cannot be reset, and cannot be used for the majority of inhalation sales, which are aerosols or non-steroidal inhalation medication. Most patients require multiple inhalers (that contain different medicines, aerosols, and varying number of doses/inhaler). Since the Advair is a sealed unit it cannot be refilled nor hold other medicines nor can the counter be set to a different number of inhalations.

There are also other prior art devices for counting doses. These devices are typically complex mechanical devices that have various mechanical means for counting dosages used or remaining in an inhaler is the cost of the devices. In order for an inhalation counter to be cost effective, the parts used in the device must be inexpensive. However, when inexpensive parts are used, there is a risk that the parts will wear out prematurely, thus causing the extra expense of replacing the part and the labor costs involved. In addition, there is also a risk when inexpensive parts are used that there be miscounts, and errors where a dose is not registered or where a multiple doses are registered when only one dose is given. Both of these situations can create serious problems. In the first instance, where a dose is not registered, the user could run out of medication at an inopportune time, such as where a replacement is not available. In the second instance, where a single dose is registered as more than one dose, the parent or other care provider could erroneously conclude that an overdose has be given to the patient. A false reading could create safety issues and cost issues that create problems for the user. Another apparatus that presented a serious health concern actually prevented the client from taking the dose if it was past due. As a result, there is a need for an improved counter for inhalers and related devices for administering medications.

### OBJECTS OF THE INVENTION

It is an object of the invention to provide a counter for use with an inhaler and the like that is reusable.

It is also an object of the invention to provide a counter for use with an inhaler and the like that is inexpensive to manufacture.

It is another object of the invention to provide a counter for use with an inhaler and the like that is accurate.

It is a further object of the invention to provide a counter for use with an inhaler and the like that is usable with a variety of different types of inhalers.

It is also an object of the invention to provide a counter for use with an inhaler and the like that has very few moving parts that can wear or break during use.

It is still another object of the invention to provide a counter for use with an inhaler and the like that is easy to use for all types of patients.

It is an object of the invention to provide a counter for use with an inhaler and the like that is easy to read.

It is an object of the invention to provide a counter for use with an inhaler and the like that will warn patient of “near empty” status.

It is an object of the invention to provide a counter for use with an inhaler and the like that will remind patients when to get a medicine refill and when to change the canister.

It is an object of the invention to provide a counter for use with an inhaler and the like that will remind patients when to take medications by means of an audio&/or visual alarm every 8 or 12 hours.

It is an object of the invention to provide a counter for use with an inhaler and the like that will prevent respiratory distress, ER visits, hospitalization, steroidal injections and patient fear.

It is an object of the invention to provide a counter for use with an inhaler and the like that will save patient money from unnecessarily disposing of inhaler with med left or from having unnecessary ER visits.

It is an object of the invention to provide a counter for use with an inhaler and the like that will save insurance companies from the expenditure of unnecessary refills and ER visits.

### BRIEF SUMMARY OF THE INVENTION

The present invention is directed to an apparatus, which is a battery, solar, mechanical, or electro/mechanically powered counter that gives digital display of either the doses taken or remaining of medicines taken through inhalers. Mechanical and electromechanical displays may also be used as well. The counter, which is interchangeable and reusable, is placed on top of the canister which sits inside an actuator. When the apparatus is depressed, the metal canister is also depressed into the actuator to dispense the inhalation medication. Each depression of the apparatus occurs simultaneously with taking a dose of medicine, the digital counter will display the number of inhalations remaining or taken depending on how the patient would like to see the read out. This device may include a more specific memory means to facilitate the monitoring of frequency, time intervals or amount of medicine dispensed or used within a given time period. Alternatively, it may be adapted to facilitate other medicines i.e. injections of insulin for diabetics or epogen injections for MS, nasal sprays, non-respiratory nasal or inhalation sprays, or even pills i.e. medications for diabetes or migraine headaches and others.

The apparatus of the present invention has several beneficial features such as the ability to:

display the number of inhalations remaining and/or utilized

allow the client to know when the medicine supply is running low

allow the client to know when to get a medicine refill

allow the client to know when to replace the metal canister

serve as reminder for patient to visit their physician

also remind the patient when the next inhalation is due

be environmentally friendly (reusable)

be universal for all aerosol inhalers-regardless of dosage, type of medicine, number of inhalations per canister, size or shape of canister or actuator

save the patient from unnecessary ER visits, hospitalizations, unnecessary injections and steroids

decrease patient fears

save patient and insurance companies money, and thus not raise insurance companies' premiums.

This apparatus is affordable, reusable, and interchangeable. It is compatible with all metal canisters and all actuators, regardless of canister or actuator size, type of aerosol medication, dosage, etc.

### BRIEF DESCRIPTIONS OF DRAWINGS

FIG. 1 is a perspective view of an inhaler with the counter of the present invention.

FIG. 2 is a side view of the inhaler of FIG. 1.

FIG. 3 is a side view of the inhaler of FIG. 2 taken along 3-3.

FIG. 3A is a view of the counter of the present invention.

FIG. 4 is cutaway view of the interior of the dispensing mechanism of FIG. 3A.

FIG. 5 is a cross sectional view of the dispensing mechanism of FIG. 3A.

FIG. 6 is a rear view of the counter of FIG. 3A.

FIG. 6A is a close up perspective of the cap and counter shown in FIG. 6.

FIG. 6B is a side perspective of FIG. 6.

FIG. 7 shows a side perspective of the cap button, 4 the sliding mechanism, 2 and the counter, 7 in relation to each other.

FIG. 8 displays the rear view of the device.

FIG. 9 displays the device as attached to the inhaler on top of the metal canister.

FIG. 10 is alternative placement of the counter.

FIG. 11 shows a detail of the cap with the concave recess.

### DETAILED DESCRIPTION OF INVENTION

The purpose of the invention is to provide an improved means of counting the number of doses taken or left in medicine taken through inhalation. Inhalation medicines are commonly taken for asthma and treating other respiratory conditions. Many of these medications contain certain steroids, and most recently insulin is being now being offered through inhalation. The medicine received by the patients is very important, and in many cases their life depends on the medication and the correct dose thereof. Thus, there is a strong need for users to know how much of their medicine they have left and when to get a refill. The present invention is directed for this purpose. The present invention is an interchangeable and reusable counter, it is placed on top of the metal canister (containing the inhalation medicine) which sits inside the actuator. When the apparatus is depressed, the metal canister is also depressed into the actuator to dispense the inhalation medication. The digital counter will display the number of inhalations remaining or taken with each depression. In addition, the present invention allows the user to decide if they want to count up as they use the medicine, or if the want to count down from the maximum number of uses contained as indicated on the medication.

FIG. 1 shows the present invention as would be used by patients. The device is placed over the inhaler, 6 that contains the metal medicine cartridge, 5. The device is secured around the inhaler by arms, 1 that extends outward from the digital counter screen, 7. FIG. 1 shows the downward movement of the sliding mechanism, 2 when the cap button, 4 is depressed, this occurs simultaneously with the release of the medication.

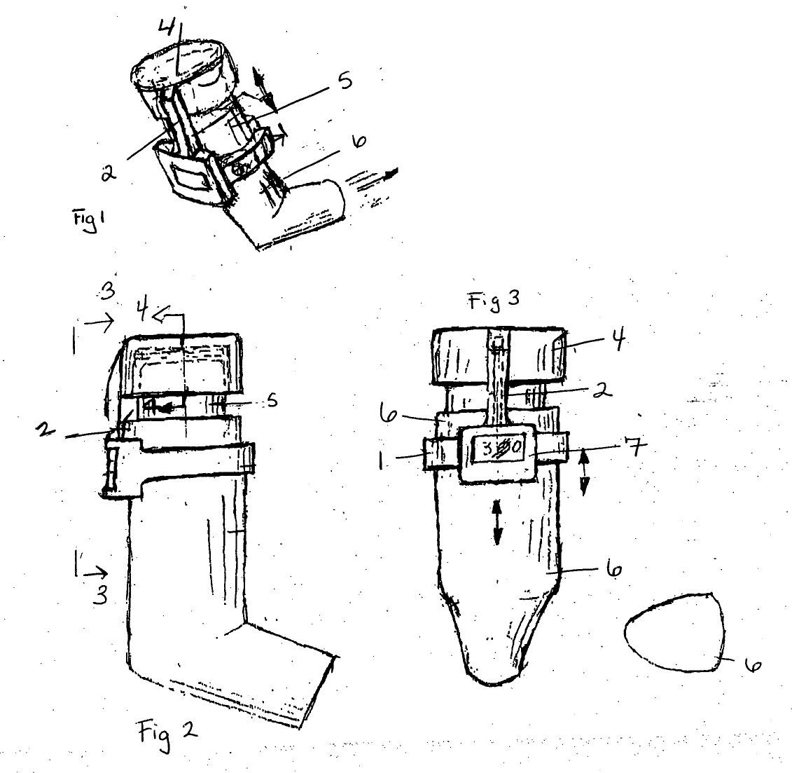

FIG. 2 displays a side perspective of the inhaler of FIG. 1. Elements 2,1,4 and 5 are detailed. The sliding mechanism, 2, refers to the range, which the device moves up and down during a depression along the inhaler, 6. The inhaler, 6, is held in place by arms that extend outward from the digital counter screen, 7. The cap button, 4, is depressed during inhalation. The button, 4, activates the digital counter screen, 7. The up and down range of the depression of the cap button, 4, is also shown. Numeral 5 refers to the top of the cartridge containing medicine.

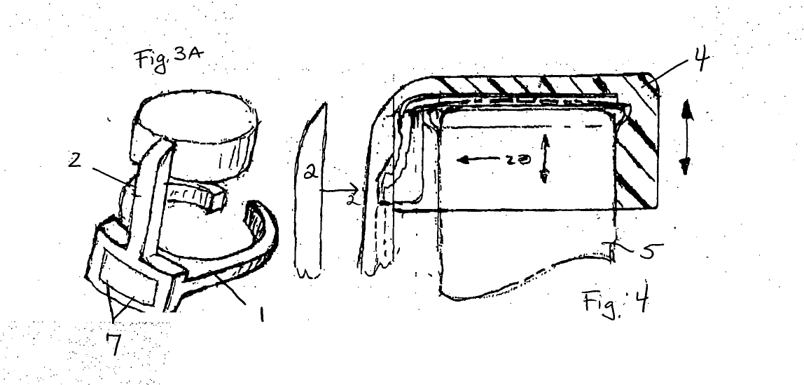

FIG. 3 displays the device as attached to the inhaler from the rear perspective showing the digital counter screen, 7 and the sliding mechanism, 2. FIG. 3 points out the inhaler with varying barrel shapes, 6 and also the cartridge containing medicine, 5.

FIG. 3A shows the device unattached to the existing dispensing means, 5 and the medication metal canister, 5. FIG. 3A illustrates the depressing mechanism or the cap button, 4, the sliding mechanism, 2 the digital counter screen; 7 and the arms that secure the device, 1.

FIG. 4 shows the cap button, 4 on top of the metal medicine canister, 5. FIG. 4 also shows the sliding mechanism, 2 that acts as the spine of the button, 4. The sliding mechanism allows the button, 4 to depress and release the medication and then to return to an un-depressed or resting state.

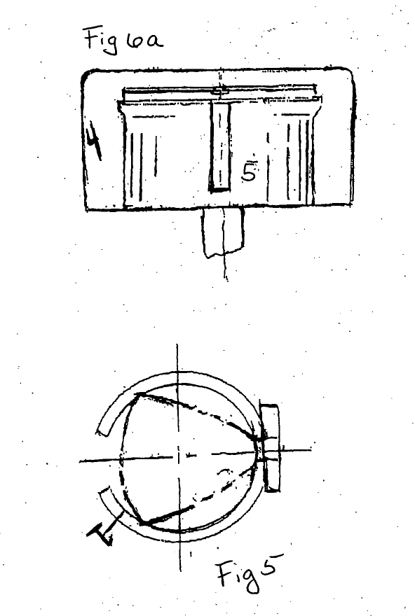

FIG. 5 shows a cross sectional view of the inhaler, 6 encircled by the arms, 1 of the device.

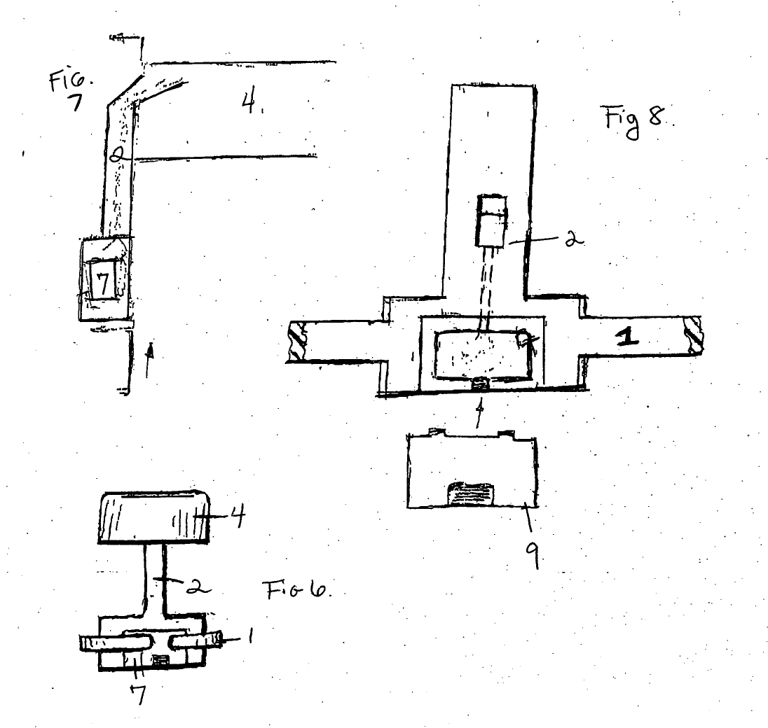

FIG. 6 shows the rear view of the device, displaying the cap button, 4 the arms, 1 as attached to the digital counter screen, 7. FIG. 6A is a close up perspective of the cap, 4 as attached to the metal canister, 5. FIG. 6B is a side perspective of FIG. 6, displaying the cap, 4 and the arms, 1 and the digital counter, 7 and also the sliding mechanism, 2.

FIG. 7 is a side perspective that shows the cap button, 4 the sliding mechanism, 2 and the digital counter screen, 7 in relation to each other.

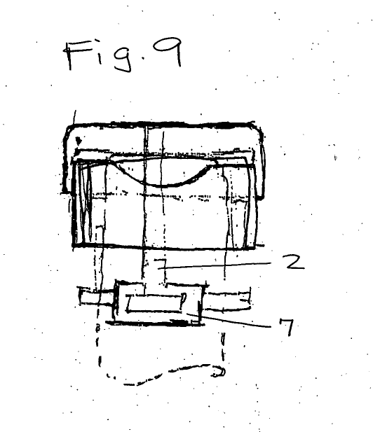

FIG. 8 displays the rear view of the device. FIG. 8 contains the sliding mechanism, 2 the digital counter screen, 7 and the arms, 1 that wrap around the inhaler for stability. Inside of the digital display the battery, 9 is contained which allows for the digital counter screen, 7 to work.

FIG. 9 displays the device, as it would function placed on top of the metal medicine canister, 5 like a cap. Although the figure shows the cap with a greater width the cap should be about the width of the metal canister so that it has a snug fit. The device is secured by the arms, 1 that wrap around the inhaler, 6.

The present invention works as such, the device is comprised of a cap button, 4 a sliding mechanism, 2 and arms, 1 that surround the inhaler, 6 and secure it. The button, 4, is pressed during inhalation; the device is allowed to move downward simultaneously with the inhaler apparatus because the sliding mechanism, 2 is adjusted to allow the button to move down along a groove. When the button, 4 is depressed, the sliding mechanism, 2 moves downward and makes contact with the battery floor, 9 and causes the counter to advance. This function repeats until all the doses of medication are finished. Then when a refill is purchased the present invention can be removed from the inhaler, 6 and a new metal medicine canister can be inserted, 5, and the present invention can be placed over the inhaler, 6.

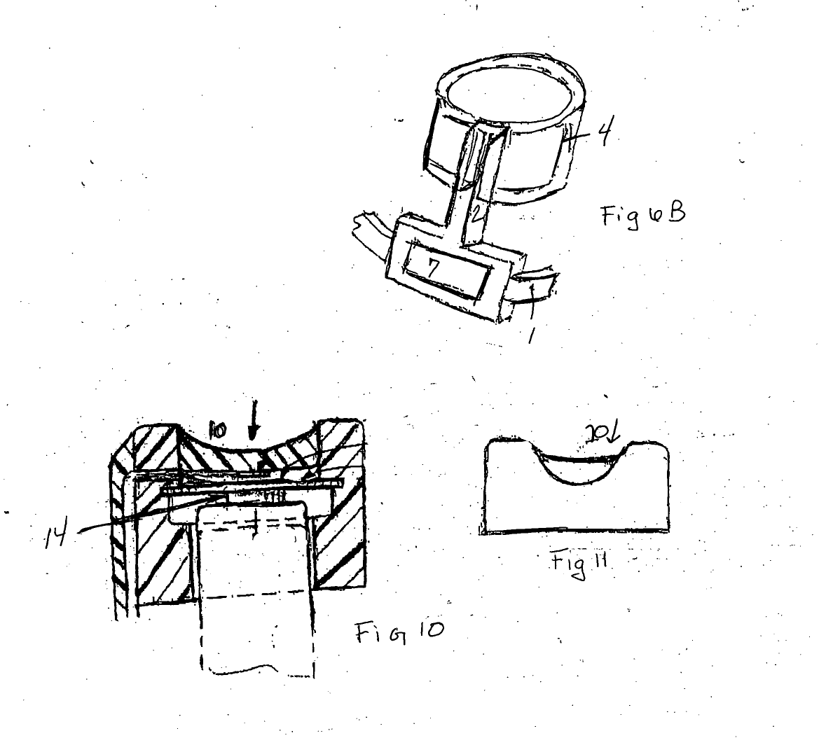

An alternative to the above invention is to place the counter on the top of the device and have a recessed cap to facilitate placement of the thumb. FIG. 10 displays a cap with a concave recess, 10. Inside of the cap is an electrical contact button, 11 an electrical contact membrane, 12 and a flexible membrane, 13. Connected to the flexible membrane, 13 is a solid push block, 14 that makes contact with the metal medicine canister, 5 and pushes the metal medicine canister, 5 downward and advances the counter.

FIG. 11 shows a detail of the cap with the concave recess, 10 for placement of the thumb.

We claim:

1. An apparatus comprising of:
A cap having a top and a bottom having hollow center;
a sliding mechanism that allows the cap to move up and down; a battery operated digital counter screen attached to the sliding mechanism; and, securing arms that extend outward.

2. An apparatus according to claim 1 in which said counter is progressed manually.

3. An apparatus according to claim 1 in which said apparatus indicated when a inhaler is almost empty.

4. An apparatus according to claim 1 in which said securing arms extend outward over an inhaler and said apparatus keeps track of how many doses of said inhaler are used.

5. An apparatus according to claim 1 in which said apparatus is used with an inhaler with a metal canister.

6. An apparatus according to claim in which said counter is solar powered.

7. An apparatus according to claim 1 in which said apparatus is depressed causing a metal canister to also be depressed into an actuator to dispense an inhalation medicine.

8. An apparatus according to claim 1 in which said apparatus is used to monitor frequency,
time intervals and amount of medicine dispensed or used.

9. An apparatus according to claim 1 in which said apparatus is reusable.

10. An apparatus according to claim 1 in which said apparatus in interchangeable.

11. An apparatus according to claim 1 in which said apparatus having an alarm to alert patients
when to take medicine

12. An apparatus according to claim 1 in which said apparatus is used with an actuator for any type of aerosol medication.
